(19) United States
(12) Patent Application Publication
(10) Pub. No.: US 2026/0326197 A1
(43) Pub. Date: Oct. 22, 2026

(54) MICROCURRENT AND PHOTOCATALYTIC GALVANIC PELLET, FABRICATION METHOD THEREFOR, AND USE THEREOF

(71) Applicants: Hong Wook PARK, Seoul (KR); Jo Dong PARK, Seoul (KR); Il Dong PARK, Seoul (KR); Mi Ja JUNG, Seoul (KR)

(72) Inventors: Hong Wook PARK, Seoul (KR); Jo Dong PARK, Seoul (KR); Il Dong PARK, Seoul (KR); Mi Ja JUNG, Seoul (KR)

(21) Appl. No.: 19/105,582

(22) PCT Filed: May 2, 2023
(86) PCT No.: PCT/KR2023/005943

§ 371 Date: Feb. 21, 2025

(30) Foreign Application Priority Data
Sep. 2, 2022 (KR) 10-2022-0111250

(51) Int. Cl. B22F 1/103; A61L 9/012; A61L 9/16; B01J 21/06; B01J 35/39; C05D 9/00; C05F 3/00; C05G 5/12

(52) U.S. Cl. CPC B22F 1/103; A61L 9/012; A61L 9/16; B01J 21/06; B01J 35/39; C05D 9/00; C05G 5/12; B22F 2302/25; C05F 3/00

(57) ABSTRACT

Disclosed herein are a galvanic pellet, a manufacturing method thereof, and a use thereof, wherein a microcurrent is generated in the pellet without a separate external current supply device, thereby inducing a photocatalytic reaction, promoting plant growth, and providing odor removal and antibacterial effects.

### TECHNICAL FIELD

### Cross Citation with Relation Application(s)

The present application claims priority to and the benefit of Korean Patent Application No. 10-2022-0111250, filed on Sep. 2, 2022, in the Korean Intellectual Property Office, the entire disclosure of which is incorporated herein by reference. The present disclosure relates to a method of fabricating a microcurrent and photocatalytic galvanic pellet that generates microcurrent without requiring a separate external power supply device, thereby exhibiting photocatalytic performance, and a use thereof.

### BACKGROUND ART

Wood pellets are produced by pulverizing wood from forests or by-products from sawmills into sawdust, then compressing the sawdust at high temperature and pressure into uniform-sized pellets.

Wood pellets are the most widely used clean woody biofuel for residential heating, public buildings, industrial facilities, and cogeneration, and a use thereof in fields other than fuel applications is also known.

As one example, Patent Document 1 discloses a technique in which sawdust is cut into pellet form, and then water is supplied to culture soil to expand the volume of wood pellets and secure plants.

As another example, wood pellets are used for handling cat excrement; however, a use thereof is not widely adopted because it is difficult to sufficiently eliminate odors. Accordingly, Patent Document 2 discloses that mixing natural bentonite sand with wood pellets can enhance odor removal and deodorizing effects However, when bentonite is used, significant dust generation causes inconvenience to users and indoor air pollution. When bentonite absorbs moisture from cat excrement, the strength thereof decreases, leading to a reduction in coagulation property. Various chemical additives are also mixed for antibacterial and deodorizing purposes, and disposing of the used sand presents an additional issue of environmental pollution.

Patent Document 3 discloses the manufacture of a compost biofilter including a filter filling material containing manure compost and wood shavings as effective components, and discloses that the biofilter may remove odorous gases generated from a composting apparatus using livestock manure. Such a biofilter is a porous product that initially removes a certain amount of ammonia gas through an odor removal effect based on gas adsorption. However, as time passes, pores become saturated, leading to a decrease in the removal rate, making it ineffective for practical applications.

Currently, recycling of wood powder and livestock manure is carried out in various fields, and most methods involve using both wood powder and livestock manure as raw materials for manufacturing fertilizer for plant cultivation. In particular, the recycling of livestock manure such as animal waste is supported as a national policy project, and many companies are engaged in the business of converting livestock manure into fertilizer. Through the use of wood powder in the conversion of livestock manure into fertilizer, livestock manure can be processed cleanly. The fertilizer produced by mixing wood powder is recognized as a highly effective organic fertilizer.
(Patent Document 1) KR 10-2012-0043293A (May 4, 2012)
(Patent Document 2) KR 10-2019-0120531A (Oct. 24, 2019)
(Patent Document 3) KR 10-2003-0085328A (Nov. 5 2003)

### DISCLOSURE

### Technical Problem

Conventional methods for removing odors and unpleasant smells have been performed by covering odors and unpleasant smells with other scents or by adsorbing odor particles. Such methods provide only a temporary effect in removing unpleasant smells and odors. However, over time, problems arise as odors re-emerge due to desorption of adsorbed particles or a decrease in the deodorization rate.

The present disclosure is intended to develop a product that enables semi-permanent and continuous deodorization regardless of time while also possessing antibacterial performance, and, as an example, provides a product manufactured to continuously generate microcurrent and exhibit photocatalytic activity, thereby achieving the above effects.

Microcurrent technology has been applied to medical industries such as wound healing and pain treatment, beauty industries such as skincare, and hygiene products, but is seldom applied to technologies related to odor removal and antibacterial performance, particularly for pet excrement odor, food waste odor, and wastewater treatment plant odor.

As a result, the present disclosure is directed to providing a galvanic pellet manufactured to enable generation of microcurrent without a separate current supply device and to be applied to various fields while allowing stimulation intensity of the microcurrent to be adjusted according to each use.

Particularly, the galvanic pellet according to the present disclosure is designed not only to be environmentally friendly and easily manufacturable by using conventionally discarded wood powder and livestock manure, but also to enable process cost reduction, mass production of various products in desired shapes, and uniform generation of microcurrent throughout the galvanic pellet. It has been confirmed that the microcurrent exhibits deodorizing and antibacterial performance, and the effect thereof is enhanced through the combined use of a material having photocatalytic activity together.

Furthermore, it has been discovered that the galvanic pellet, in addition to the aforementioned deodorizing and antibacterial performance, is also effective in promoting plant growth due to the continuous generation of microcurrent.

Embodiments of the present disclosure are intended to provide a galvanic pellet, a fabrication method thereof, and a use thereof.

### Technical Solution

The present disclosure may provide a galvanic pellet that generates microcurrent and exhibits photocatalytic activity.

The galvanic pellet may include: an organic powder; a first metal powder and a second metal powder that have different materials; and a binder.

The first and second metal powders may generate microcurrent by electrochemical reaction due to an electric potential difference under presence of an electrolyte, and each include one selected from Al, Zn, Fe, Ni, Sn, Hg, Ag, Pt, Au, and combinations thereof.

The organic powder may be obtained by pulverizing one or more types of by-products selected from the group consisting of food waste, livestock waste, woody waste, agricultural by-products, and forestry by-products.

The binder may be a biodegradable binder of a single type selected from the group consisting of lignin, starch, collagen, gelatin, hyaluronic acid, chitosan, alginic acid, beta-glucan, cellulose, carrageenan, pluronic, and combinations thereof.

Furthermore, the galvanic pellet may further include, as an additive, one selected from the group consisting of a filler, a lubricant, and combinations thereof.

The filler may include titanium dioxide having photocatalytic activity.

The present disclosure may provide a method of fabricating a galvanic pellet. The method may include mixing an organic powder with a first metal powder and a second metal powder that have different materials, a binder, an additive and performing a pellet forming process by introducing the mixture into a pellet forming unit.

Furthermore, the present disclosure may provide a use for the galvanic pellet in removing odors from pet litter, a food waste disposer, or an adsorbent for a wastewater treatment plant.

Additionally, the present disclosure may provide a use for the galvanic pellet that is applied to soil to promote plant growth.

In addition, the present disclosure may provide a use for pet litter including the galvanic pellet.

Furthermore, the present disclosure may provide a use for a plant growth promoter pellet including the galvanic pellet.

### Advantageous Effects

A galvanic pellet according to the present disclosure spontaneously generates microcurrent without a separate external current supply device, producing hydrogen and ozone as products of an electrochemical reaction, thereby exhibiting deodorizing and antibacterial performance. Accordingly, the galvanic pellet can be used as a pellet for pet litter, food waste odor removal, wastewater treatment plant odor removal, and plant growth promotion.

The galvanic pellet can rapidly absorb pet urine and deodorize odors generated from the urine through microcurrent while also exhibiting an antibacterial effect. As a result, compared to conventional pet litter, the occurrence of odors can be reduced, and the processing time can also be shortened.

The foregoing galvanic pellet generates microcurrent, which aids in plant growth.

The galvanic pellet according to the present disclosure may be an environmentally friendly technology that enables resource utilization and industrialization by reusing forestry and timber waste, such as sawdust that is conventionally discarded, or livestock manure.

### BRIEF DESCRIPTION OF DRAWINGS

FIG. 1(a) is a photograph showing a galvanic pellet in a dry state, and FIG. 1(b) is a photograph showing lump formation when in contact with an electrolyte.

FIG. 2 is a photograph related to measurement of total organic carbon (TOC) in wastewater using the galvanic pellet.

FIG. 3 is a photograph showing the growth of lettuce based on the application of the galvanic pellet.

FIG. 4 is a photograph showing the state of lettuce after being washed with water.

### BEST MODE

An embodiment of the present disclosure may provide a galvanic pellet including: an organic powder; a first metal powder and a second metal powder that have different materials; and a binder, wherein the first and second metal powders generate microcurrent due to an electric potential difference under the presence of an electrolyte.

Another embodiment of the present disclosure may provide a method of fabricating the galvanic pellet, the method including mixing an organic powder with a first metal powder and a second metal powder that have different materials, and a binder, and performing a pellet forming process by introducing the mixture into a pellet forming unit, wherein the first and second metal powders generate microcurrent due to an electric potential difference in the presence of an electrolyte.

### MODE FOR DISCLOSURE

The present disclosure provides a galvanic pellet applicable to various fields by generating microcurrent in an electrolyte environment without a separate external current supply device and supplying bandgap energy required for a photocatalytic reaction, thereby enabling additional photocatalytic activity.

The galvanic pellet according to the present disclosure is designed based on the structure of a galvanic cell, wherein an anode and a cathode induce the generation of microcurrent through an oxidation-reduction reaction caused by an electric potential difference in the presence of an electrolyte.

The known zinc-silver galvanic cell undergoes the following electrochemical reactions.

Eo=+0.80 V Ag⁺ (aq)+e⁻¹--->Ag(s) reduction

Eo=−0.76 V Zn²⁺(aq)+2e⁻¹--->Zn(s) oxidation

Ecell=+1.56 V 2Ag⁺(aq)+Zn(s)--->2Ag(s)+Zn²⁺(aq)

As shown in the above equation, Zn that emits electrons and loses electrons ionizes into Zn²⁺ (cation), while Ag that gains electrons becomes an anode (+), and microcurrent is generated in this process.

In an electrochemical reaction, the greater the ionization tendency, the more easily electrons are lost, making it easier to become a cation (oxidation). When Zn and Ag are combined as shown below, Zn becomes a cathode, and Ag becomes an anode. However, when Ag and Au are combined, Ag becomes the cathode, and Au becomes the anode.

### (Ionization Tendency)

K>Ca>Na>Mg>Al>Zn>Fe>Ni>Sn>Pb>H>Cu>Hg>Ag>Pt>Au

A galvanic cell operates based on an electric potential difference between two types of metals, and even the same metal can serve as either the anode or the cathode. In this case, when metals with a significant difference in ionization tendency are combined (i.e., the greater the electric potential difference), the intensity of the generated microcurrent also increases.

The aforementioned electric potential difference generates microcurrent, which is induced more rapidly in the presence of an electrolyte.

As shown in the following reaction equation, metal (M) reacts with the electrolyte and water, generating ionized metal along with ozone and hydrogen.

M²⁺+H₂O+NaCl (electrolyte)--->M⁺+H₂+O₃+(Na⁺/Cl⁻)

The hydrogen generated by the above reaction equation reacts with ammonia, as shown in the following reaction equation, and is converted into an ammonium salt. Through the aforementioned process, ammonia, which causes odor generation, can be removed.

Deodorization performance: 2NH₃+H₂--->2NH₄

Furthermore, the ozone (O₃) generated by the above reaction equation exhibits antibacterial performance.

Moreover, as shown in the above reaction equation, the ozone decomposes ammonia and trimethylamine, which cause odor generation.

In nature, ozone possesses the second strongest oxidizing power after fluorine, with the oxidizing power seven times that of chlorine, and thus, is widely used in various fields such as oxidation, sterilization, decolorization, and deodorization. Since ozone ultimately decomposes into oxygen, it does not cause secondary pollution and is environmentally friendly.

As described above, the galvanic pellet of the present disclosure possesses excellent deodorization performance and antibacterial performance. Compared to conventional adsorbents or antibacterial agents, which are used temporarily or degrade in performance over time, the galvanic pellet of the present disclosure can continuously generate microcurrent along with hydrogen and ozone through the electrochemical reactions, thereby maintaining the effectiveness with negligible degradation over time.

Furthermore, since the conditions for generating microcurrent, hydrogen, and ozone involve an electrolyte containing water, no additional composition or device is required, resulting in no significant cost to achieve the above effects.

In addition, in the present disclosure, microcurrent is not generated by an external current supply device but is induced by a potential difference between metals with different electrical properties, enabling continuous and nearly uniform microcurrent supply through an electrochemical oxidation-reduction reaction.

The galvanic pellet capable of supplying microcurrent through a potential difference includes: an organic powder; metal powders that include two or more different metal materials and are capable of generating a potential difference; a binder; a filler; and a lubricant.

Each composition is described in detail below.

The organic powder is obtained by pulverizing one or more types of by-products selected from the group consisting of food waste, livestock waste, woody waste, agricultural by-products, and forestry by-products.

The food waste may include various waste residues generated in the food industry, as well as scraps from seafood processing, canning factories, and fish markets. The livestock waste may include manure from cattle, pigs, and chickens. The woody waste may include wood materials from the construction industry, bark from logs in processing plants, sawdust, and wood chips. The agricultural by-products may include rice straw, barley straw, and rice husks. The forestry by-products may include by-products generated from roadside tree or lawn maintenance, or wood waste produced from logging operations.

According to an embodiment, the organic powder may be wood powder and/or livestock manure powder.

The wood powder refers to finely ground material derived from wood, branches, or roots, obtained by crushing waste wood or by pulverizing sawdust generated during the sawing process into fine powder using a pulverizer, followed by sieving. The wood powder contains numerous resulting in extremely lightweight properties and high cost-effectiveness. For example, the wood powder may be derived from pine or juniper.

The livestock manure powder refers to a powderized material obtained by fermenting and drying livestock excrement along with auxiliary materials. The livestock manure powder can supply nitrogen (N) and phosphorus (P) to crops. The livestock manure powder may be derived from one or more selected from cattle manure, swine manure, and poultry manure, and other types of livestock manure may also be used.

The wood powder and livestock manure powder are manufactured in pellet form, and the particle size is preferably limited to ensure compatibility with other components. In an embodiment, the wood powder and livestock manure powder preferably have an average particle diameter ranging from 10 mesh (2 mm) to 45 mesh (0.354 mm) or from 18 mesh (1 mm) to 35 mesh (0.5 mm).

The wood powder and livestock manure powder can be used alone or in combination, and when mixed, a content ratio may vary depending on the intended use.

For example, when used as pet litter (which will be described below), such as cat litter, the wood powder, which has pores and excellent absorbency, may be used alone to enhance feces absorption or may be mixed with a certain amount of livestock manure powder. On the other hand, for plant cultivation, the livestock manure powder may be used alone to ensure a sufficient supply of nitrogen and phosphorus or may be mixed with a certain amount of wood powder. In the case of single use, there is an advantage of enhancing the effects of each of the wood powder and the livestock manure powder. However, for forming into pellet form and ensuring a certain level of strength, combining both materials is more advantageous.

Specifically, in the galvanic pellet of the present disclosure, the wood powder and the livestock manure powder are each used in an amount of 50 wt % to 95 wt % within a total of 100 wt % of waste powder. For example, when used as pet litter, the wood powder is preferably used in an amount of 85 wt % to 95 wt %, whereas for plant applications, the livestock manure powder is preferably used in an amount of 80 wt % to 90 wt %, and as needed, a mixture of both may also be used.

The waste powder is contained in an amount of 90 wt % to 95 wt % within the total composition of the galvanic pellet, which sums to 100 wt %. If the content is below the aforementioned range, it becomes difficult to manufacture the galvanic pellet, whereas if it exceeds the range, the relative content of other components decreases, making it difficult to achieve the desired physical properties and effects.

The metal powder includes different metal materials, e.g., at least two types of different metal materials capable of generating an electric potential difference.

According to an embodiment, the metal powder includes two types of metal particles, i.e., a first metal particle and a second metal particle. The first and second metal particles are made of different metal materials, and a potential difference is generated due to a difference in their ionization tendencies, thereby generating microcurrent.

The first metal particle and the second metal particle each include a metal material and may be one selected from Al, Zn, Fe, Ni, Sn, Cu, Hg, Ag, Pt, Au, and combinations thereof. The first metal particle and the second metal particle include different metal materials to generate a potential difference. In this case, the combination of metals includes not only an alloy form of two or more metals but also a simple mixture or a core-shell structure.

According to an embodiment, when the first metal particle functions as an anode, the first metal particle may be Ag, Pt, Au, or a combination thereof, and in this case, the second metal particle functions as a cathode and may be Al, Zn, or a combination thereof.

According to another embodiment, when the first metal particle functions as a cathode, the first metal particle may be Al, Zn, or a combination thereof, and in this case, the second metal particle functions as an anode and may be Ag, Pt, Au, or a combination thereof.

Specifically, the metal powder may have a particle size ranging from 1 nm to 20 μm. Preferably, the metal powder may be a nanoparticle with a size ranging from 1 μm to 20 μm, and may have a spherical, oval, rod-like, hollow, or amorphous shape. In this case, surface-treated particulate metal powder, such as hydrophobically treated metal powder, may be used, allowing the application thereof regardless of surface properties such as hydrophobicity or hydrophilicity of metal powder.

The metal powder is contained in an amount of 0.1 wt % to 10 wt % within the total composition of the galvanic pellet, which sums to 100 wt %. If the content is below the aforementioned range, microcurrent generation is insufficient, making it impossible to achieve the intended effects. Conversely, if the content exceeds the range, aggregation may occur, making it difficult to generate uniform microcurrent.

The binder serves to combine the remaining components, promote cohesion during the forming process, and maintain the strength of the galvanic pellet.

As the binder, one biodegradable and eco-friendly binder of a single type selected from the group consisting of lignin, starch, collagen, gelatin, hyaluronic acid, chitosan, alginic acid, beta-glucan, cellulose, carrageenan, pluronic, and combinations thereof may be used. Compared to conventional urethane or acrylic binders, the aforementioned materials are biodegradable, making the disposal of used galvanic pellets significantly easier.

Lignin and starch are preferable among the binder materials. The lignin is not particularly limited and may be obtained by hydrolyzing lignocellulosic biomass with acid. Furthermore, starch, as an eco-friendly material, may be selected from corn starch, potato starch, sweet potato starch, cassava starch, Takao starch, or wheat starch.

The binder is contained in an amount of 0.5 wt % to 5 wt % within the total composition of the galvanic pellet, which sums to 100 wt %. If the content of the binder is below the aforementioned range, the binding strength is relatively low, making the pellet forming process difficult or causing the manufactured pellet to break easily. Conversely, if the content of the binder exceeds the aforementioned range, the pores of the galvanic pellet may become clogged, hindering the approach of the electrolyte (such as bodily fluids or water) to the metal powder, resulting in insufficient or significantly reduced microcurrent generation.

As the filler, one selected from the group consisting of porous silica, titanium dioxide, bentonite, zeolite, clay, mica, kaolin, diatomaceous earth, perlite, activated carbon, and combinations thereof may be used. Among the aforementioned materials, titanium dioxide may be used due to excellent photocatalytic performance, adsorption capacity, coagulation ability, and deodorizing performance thereof. As the filler, a material having an average particle diameter ranging from 1 nm to 10 μm are used.

The filler is contained in an amount of 0.1 wt % to 5 wt % within the total composition of the galvanic pellet, which sums to 100 wt %. If the content is below the aforementioned range, the above-described effects cannot be achieved. Conversely, if the content exceeds the aforementioned range, the pores of the galvanic pellet may become clogged, or the galvanic pellet may be easily broken. Therefore, the filler is appropriately used within the aforementioned range.

In an embodiment, the filler according to the present disclosure may include titanium dioxide having photocatalytic activity.

The photocatalytic activity not only oxidizes and reduces pollutants but also has the advantage of simultaneously exhibiting a sterilizing effect. The photocatalytic activity follows a fundamental reaction mechanism in which valence band holes (h+) react with water to generate hydroxyl radicals (OH), and conduction band photoexcited electrons undergo a reduction reaction, reducing oxygen in the air to form reactive oxygen species (superoxide anion O2−).

Photocatalytic activity: H₂O+H⁺->·OH+H⁺

O₂+e⁻->O²⁻

The reactive species formed through the foregoing process directly react with organic compounds or the like, thereby not only decomposing and removing impurities through strong oxidation-reduction reactions but also eliminating ammonia, which causes odors, while exhibiting antibacterial properties.

The titanium dioxide according to the present disclosure may be titanium dioxide produced from a photocatalyst composition including 10 wt % to 30 wt % titanium tetra isopropoxide (TTIP), 20 wt % to 40 wt % ethanol, 1.5 wt % to 2.0 wt % acid, 0.1 wt % to 1.0 wt % thickener, 0.5 wt % to 1.5 wt % dispersant, and water as a remainder.

A cellulose-based compound such as hydroxyl propyl cellulose (HPC) or hydroxyl ethyl cellulose (HEC) may be used as the thickener. A surfactant may be used as the dispersant.

The photocatalyst composition is reacted to produce titanium dioxide having an anatase crystal structure, and the titanium dioxide thus produced has a crystal size ranging from several nanometers to several tens of nanometers, for example, 1 nm to 90 nm. Nano-sized titanium dioxide, due to the photocatalytic effect thereof, is highly effective in removing harmful substances such as ammonia, triethanolamine, and acetaldehyde, which cause odors.

The lubricant is added to enhance bonding or affinity between the aforementioned components during the component forming process, to facilitate mixing, to reduce frictional heat generated during the forming process, thereby preventing thermal decomposition, and to enable smooth forming operations.

As the lubricant, one or more selected from the group consisting of stearates, palmitates, laurates, and combinations thereof, which are natural substances with high environmental compatibility, may be used. More preferably, calcium stearate, zinc stearate, or a mixture thereof may be used.

The lubricant is contained in an amount of 1 wt % to 5 wt % within the total composition of the galvanic pellet, which sums to 100 wt %. If the content is below the aforementioned range, sufficient lubrication cannot be achieved during a product forming process. Conversely, if the content exceeds the aforementioned range, defects may form inside the galvanic pellet, or flow-marks or the like may occur during the product forming process, compromising the aesthetic appearance of a product.

The galvanic pellet according to the present disclosure is manufactured using a pellet forming apparatus.

According to an embodiment, the pellet forming apparatus includes a hopper, a mixer, a pellet forming unit, and a controller. The foregoing components of the pellet forming apparatus are connected by a lifting elevator and/or a transfer conveyor.

First, powder containing wood powder and livestock manure powder stored in the hopper is fed into the mixer. Metal powder comprising two or more different metal materials capable of generating a potential difference, a binder, a filler, and a lubricant are added to the powder and then mixed.

Next, the mixed powder from the mixer is input into the pellet forming unit to perform pellet formation.

The pellet formation may be carried out using an extrusion-type forming unit that shapes the mixture by applying high pressure thereto, but is not limited thereto. The pellet forming unit applies pressure to the mixture using a driving device, such as a compression wheel inside a ring die formed with a pellet hole, and forms the galvanic pellet by pressing and discharging (extruding) the mixture in a horizontal direction through an extrusion hole in a metal plate.

The foregoing process may be controlled by the controller.

The galvanic pellet produced through the above process may undergo post-processing, such as chopping or milling, if necessary, to be manufactured into various shapes, and thereafter, may be input into a screen-type separator to filter out fine particles, followed by a sorting and packaging step in which produced galvanic pellets are packaged in a predetermined weight for commercialization.

As needed, a dryer may be installed after the pellet forming unit to perform an additional drying step for adjusting the moisture content of the galvanic pellet

The adjustment of the moisture content is performed to improve storage stability, and the galvanic pellet may be dried after fabrication so that the moisture content thereof is 15 wt % or less, preferably 1 wt % to 15 wt %, and more preferably 5 wt % to 15 wt %.

The galvanic pellet according to the present disclosure, fabricated as described above, has a diameter ranging from 1 mm to 10 mm, preferably from 2 mm to 5 mm, and a length ranging from 2 mm to 5 mm or 10 mm to 15 mm. Furthermore, the compressive strength is 900 g or more, and the density ranges from 1.05 g/cm3 to 1.15 g/cm3. The galvanic pellet according to the present disclosure has various shapes depending on the use thereof. For example, the galvanic pellet encompasses all three-dimensional forms, including spherical, oval, cylindrical, columnar, cubic, rectangular parallelepiped shapes, and solid or hollow rods or cylinders with arbitrary cross-sectional areas. Furthermore, the galvanic pellet also includes irregular shapes such as flat chips, flakes, double chips, and wave-cut forms.

For example, 10 g of pellets includes 10 to 500 pellets, preferably approximately 20 to 100 pellets per 10 g, and more preferably approximately 25 to 80 pellets.

Although it is preferable for the galvanic pellets to have approximately the same size, it is also possible to use a mixture of pellets with different shapes, sizes, and formulations.

Parameters related to the pellet forming process include the composition of raw materials, particle size, compression ratio, and moisture content. The composition and particle size follow the aforementioned descriptions.

The galvanic pellet according to the present disclosure has a compression ratio (pellet length/pellet diameter) ranging from 5 to 7. To satisfy the aforementioned compression ratio, process parameters such as an rpm of a pelletizer, a working width of a compression unit, a thickness of a die, a relief hole, a guide angle, depth, and shape of a raw material inlet, a design of a main shaft, and a selection of bearings can be adjusted, and the foregoing parameters can be appropriately designed by a person skilled in the art.

The galvanic pellet according to the present disclosure is effective in odor removal, antibacterial effects, and plant growth due to microcurrent generation and additional photocatalytic activity and can be applied to various fields where such applications are possible.

According to an embodiment, the galvanic pellet of the present disclosure may be used as pet litter.

Pet litter is used for handling pet excrement and is commonly known as ‘cat litter’. In the case where the galvanic pellets of the present disclosure are used in a pet toilet, the galvanic pellets spread to a thickness of 2 cm to 3 cm. The galvanic pellets that come into contact with pet urine coagulate and then disintegrate. The disintegrated pellets are discarded.

Pets include cats, dogs, hedgehogs, rabbits, hamsters, lizards, and parrots, and the present disclosure may be applied to other pets as well.

When the galvanic pellets are used as pet litter, if pet urine, particularly cat urine, comes into contact with the galvanic pellets, the pellets coagulate and form lumps. The galvanic pellet according to the present disclosure rapidly absorbs moisture, allowing for immediate coagulation.

Upon contact with urine, the galvanic pellet immediately generates microcurrent, producing hydrogen and ozone, which enable deodorization of ammonia, triethylamine, and nitrogen dioxide (NO₂) generated from pet urine, particularly cat urine. Additionally, the antibacterial effect against bacteria caused by urine contributes to maintaining a sanitary pet toilet environment.

Furthermore, the galvanic pellet is fabricated in pellet form, maintaining a uniform particle shape, thereby preventing fine powder from adhering to the paws of a cat, minimizing dust generation, and reducing the amount of litter scattered outside the pet toilet. In addition, urine is rapidly absorbed, maintaining the interior of t the pet toilet fresh always and reducing the likelihood of conditions such as conjunctivitis, allergies, or paw pad cracking caused by litter desertification.

The galvanic pellet of the present disclosure can be used alone or in combination with conventional bentonite litter, tofu litter, wood pellets, or the like. When used in combination, the galvanic pellet can provide both the deodorization and antibacterial effects along with the benefits of each type of litter material.

According to an embodiment, when used as cat litter, the galvanic pellet may be manufactured and used in the form of a spherical pellet, a cylindrical pellet, or a columnar pellet. The spherical pellet has a size and shape similar to existing bentonite litter, while the cylindrical or columnar pellet has a size and shape similar to tofu litter or a wood pellet, allowing cats to use the pellets without having resistance.

Furthermore, the galvanic pellet may also be used as bedding for rabbits, hamsters, and the like, in which case the galvanic pellet may be in the form of a chip or flake.

The galvanic pellet of the present disclosure contains neither harmful substances nor additives when used as pet litter, providing a safe environment for pet health. In addition, highly moisture absorbent natural materials with excellent deodorizing capability are used, so that after use, the pellet can be used as fertilizer for potted plants.

According to another embodiment, the galvanic pellet of the present disclosure may be used for removing odors from food waste.

Food waste disposal machines include all of the followings: storage containers for collecting food waste before disposal in household, commercial, or industrial settings, apparatuses for pulverizing food waste, apparatuses for drying food waste, and apparatuses for pulverizing food waste after drying.

Food waste always contains moisture due to water from food or ingredients, making it highly suitable for the use of galvanic pellets.

For example, adding a certain amount of galvanic pellets of the present disclosure to a container used for collecting food waste at home can eliminate odors generated from the food waste. When left for a long time or for a short period during summer, food waste can develop mold and decay. However, simply adding the galvanic pellets provides an antibacterial effect that prevents the food waste from rotting. Particularly, since the galvanic pellet of the present disclosure is made from natural materials, there is no need to separately remove the galvanic pellets when recycling food waste.

According to another embodiment, the galvanic pellet of the present disclosure may be used in wastewater treatment plants.

Through wastewater treatment, various harmful substances in wastewater, such as organic pollutants and heavy metals, are removed from wastewater. Here, various environmentally friendly technologies such as the addition of adsorbents like activated carbon, ozone oxidation, and Fenton oxidation are utilized. The galvanic pellet according to the present disclosure may be used in an adsorbent addition step or an ozone oxidation step of wastewater treatment, where the galvanic pellet is added in the same manner as activated carbon. As a result, the hydrogen and ozone generated by the microcurrent produced through the electrochemical reaction of the galvanic pellet enable both odor removal and an antibacterial effect in the wastewater.

The galvanic pellets may be used in a spherical shape, like activated carbon, or in a cylindrical shape or the like to increase the surface area.

According to another embodiment, the galvanic pellet of the present disclosure may be used as a soil additive for plant growth.

The plants in the present disclosure include plants with differentiated roots, stems, and leaves, where these parts are distinctly identifiable, including leafy vegetables, fruit-bearing vegetables, root vegetables, flowers, fruit trees, and grains. The plants include germinated, partially germinated, sprouted, or partially sprouted microgreens, seeds, roots, and sprouts.

Plants perform photosynthesis, by which microcurrent of approximately 4 μA flows. The intensity of current varies depending on the type and condition of the plant, and healthier plants exhibit higher microcurrent levels. As shown in the following test examples, the galvanic pellet of the present disclosure may generate microcurrent of approximately 10 μA to 70 μA, stimulating plants through the microcurrent. Through the foregoing stimulation, the generated microcurrent can supplement the energy obtained during photosynthesis, promoting the growth of plant roots, stems, fruits, and leaves. In the case of seeds, germination is accelerated, and the number of sprouts increases, leading to an expected improvement in yield. Particularly, it is difficult to control the intensity of the electrical stimulation current when promoting plant growth through electrical stimulation. However, since the galvanic pellet according to the present disclosure generates microcurrent, the galvanic pellet can be applied without such difficulties.

The galvanic pellet according to the present disclosure may be used by being mixed with soil where plants are grown or by being spread on the soil surface. A minimal amount of water is necessary for plant growth, and various nutrients or fertilizers are used to provide nourishment. Such water, as well as nutrients and fertilizers, contain electrolyte components that can induce microcurrent generation in the galvanic pellet.

The galvanic pellet may be used in a spherical shape or in the form of a chip or flake.

### Embodiment

The following describes embodiments of the present disclosure. The embodiments are provided to describe the present disclosure in more detail, and it will be apparent to those of ordinary skill in the art that the scope of the present disclosure is not limited to these embodiments.

### Production Example 1: Production of Photocatalytic Titanium Dioxide

30 wt % TTIP, 20 wt % ethanol, 2.0 wt % hydrochloric acid, 0.50 wt % thickener (hydroxylpropyl cellulose), 1 wt % dispersant, and the remaining amount of water were added to a reactor. The mixture was then reacted for 4 hours to produce titanium dioxide nanoparticles. The produced titanium dioxide nanoparticles (50 nm) were used after drying, and analysis by X-ray spectroscopy confirmed that the nanoparticles had an anatase phase.

### Embodiment and Comparative Example: Fabrication of Galvanic Pellet

As an organic waste material, pine sawdust was purified to remove foreign substances, pulverized to a size of 1 mm, and then dried (moisture content: 18 wt %). Furthermore, manure with an average diameter of 1 mm was purchased and used.

After adding a composition shown in Table 1 to the mixer, small cylindrical galvanic pellets were fabricated using Company A's M-768 machine under a pressure of 750 kg/cm³ to 1,000 kg/cm³. Here, Zn (average particle diameter=5 μm), Ag (average particle diameter=1 μm), and titanium dioxide (average particle diameter=50 nm) were used. The moisture content of the fabricated galvanic pellets was found to be 12% or less, and each galvanic pellet had a size of 3 mm×5 mm.

TABLE 1

| | | Embodiment | Embodiment | Embodiment | Embodiment | Embodiment | Comparative |
|---|---|---|---|---|---|---|---|
| Composition (wt %) | | 1 | 2 | 3 | 4 | 5 | example 1 |
| Organic | Sawdust | 85.5 | 80 | 70 | 65 | — | 91.5 |
| Powder | Manure | — | 5.5 | 15.5 | 25.5 | 85.5 | — |
| Metal | Zn | 3 | 3 | 3 | 3 | 3 | — |
| Powder | Ag | 3 | 3 | 3 | 3 | 3 | — |
| Binder | Starch | 1.5 | 1.5 | 1.5 | 1.5 | 1.5 | 1.5 |
| Filler | Titanium | 5 | 5 | 5 | 5 | — | 5 |
| | Dioxide | | | | | | |
| Lubricant | Calcium | 2 | 2 | 2 | 2 | 2 | 2 |
| | Stearate | | | | | | |

### Test Example 1: Moisture Absorption (Microcurrent Verification)

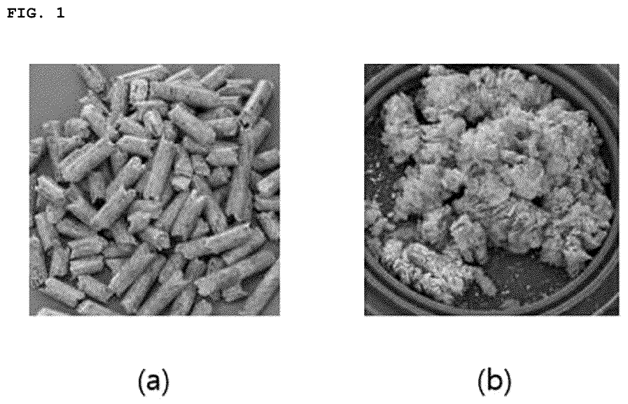

FIG. 1(a) is a photograph showing a galvanic pellet in a dry state, and FIG. 1(b) is a photograph showing lump formation when saline solution is poured. After pouring the saline solution, as shown in FIG. 1(b), a microcurrent measurement device was used to verify whether a microcurrent was generated, and the results are presented below. Each value represents an average obtained from 50 galvanic pellets.

TABLE 2

| | Microcurrent Generation | Value (μA) |
|---|---|---|
| Embodiment 1 | Generated | 25 |
| Embodiment 2 | Generated | 28 |
| Embodiment 3 | Generated | 33 |
| Embodiment 4 | Generated | 42 |
| Embodiment 5 | Generated | 40 |
| Comparative | Not Generated | 0 |
| Example 1 | | |

From the table above, it was confirmed that the galvanic pellets fabricated according to the present disclosure generated microcurrent.

### Test Example 2: Antibacterial Test

To evaluate antibacterial activity, Escherichia coli was cultured on a Nutrient agar plate in a Petri dish using an incubator at 37° C. The lump-shaped galvanic pellets shown in FIG. 1(b) were placed on multiple disc-shaped filter papers. The filter papers with the pellets were then placed on a solid agar medium inoculated with a test strain, cultured, and the size of a clear zone (growth inhibition halo) around each disc was measured. For comparison of antibacterial effects, only Escherichia coli was cultured without adding an antibacterial and deodorizing composition, and this was set as Control Example 1. Here, the larger the clear zone, the stronger the antibacterial activity, and the results are shown in the table below.
(Notation based on clear zone size: −; 0 mm, +: 5 mm or less, ++: 6 mm to 10 mm, +++: 11 mm to 15 mm, ++++: 15 mm to 20 mm, +++++: 20 mm or more)

TABLE 3

| | Escherichia coli |
|---|---|
| Embodiment 1 | ++++ |
| Embodiment 2 | +++ |
| Embodiment 3 | +++ |
| Embodiment 4 | +++ |
| Embodiment 5 | ++++ |
| Comparative | − |
| Example 1 | |
| Control | − |
| Example | |

From the table above, it can be seen that the galvanic pellet of the present disclosure exhibits an antibacterial effect.

### Test Example 3: Deodorization Effect

A deodorization test was conducted for ammonia (NH₃), triethylamine ((CH₃)₃N), and nitrogen dioxide (NO₂), which are components of cat excreta.

First, the deodorization test was conducted by placing 100 g of the galvanic pellets fabricated in Embodiment 1 into a reaction chamber having a size of 6 L, sealing the reaction chamber, and injecting test gas at an initial concentration of 50 μmol/mol into the reaction chamber. The concentration of the test gas was then measured at 0, 30, 60, and 120 minutes. Here, the concentration of the test gas was measured in accordance with KS I 2218. Furthermore, during the test, the temperature was maintained at 23±5° C., and the humidity was maintained at 50±10%. The removal rate of the test gas at each time interval was calculated based on Equation 1 below.

TABLE 4

| Category | Time (min) | Deodorization Rate (%) |
|---|---|---|
| Ammonia (NH₃) | 0 min | 0 |
| | 30 min | 99 |
| | 60 min | 99 |
| | 90 min | 98 |
| | 120 min | 98 |
| Triethylamine ((CH₃)₃H) | 0 min | 0 |
| | 30 min | 75 |
| | 60 min | 85 |
| | 90 min | 95 |
| | 120 min | 95 |
| Nitrogen Dioxide (NO₂) | 0 min | 0 |
| | 30 min | 65 |
| | 90 min | 75 |
| | 60 min | 80 |
| | 90 min | 95 |
| | 120 min | 97 |

From the table above, it can be confirmed that the galvanic pellet of the present disclosure exhibited an immediate deodorization effect upon contact with ammonia, triethylamine, and nitrogen dioxide, which are components of pet excreta, and the effect persisted even after 120 minutes. The results demonstrate that the galvanic pellet of the present disclosure exhibits excellent effectiveness in removing odors, such as pet body odor and excreta odor.

### Test Example 4: Effect for Wastewater Odor Removal

The effect for wastewater odor removal was confirmed by measuring the total organic carbon (TOC) in wastewater using the galvanic pellets of the present disclosure.

500 ml of wastewater (750 ppm of TOC) was prepared by mixing 500 ml of raw water with detergent (0.5 g or less).

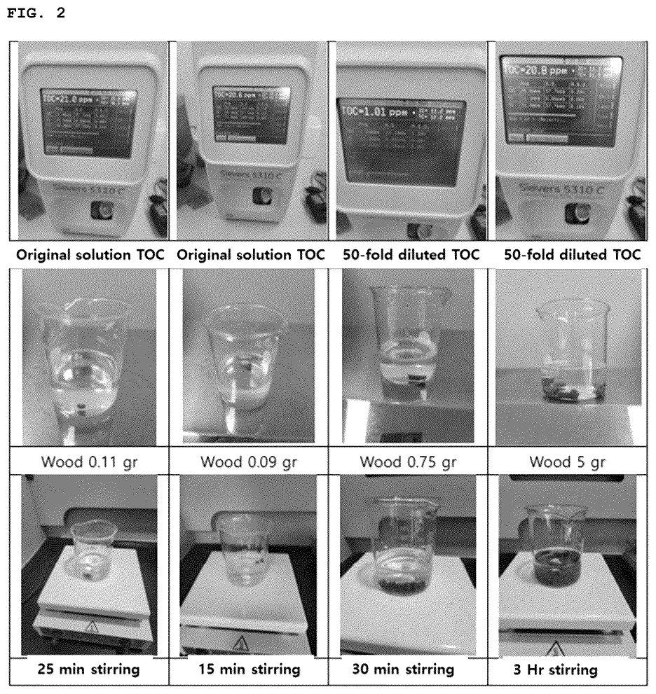

100 ml of wastewater was added to a beaker, followed by the addition of the galvanic pellets fabricated in Embodiment 1, and the mixture was stirred at 150 rpm. A sample was then collected, and TOC was measured. The results are shown in the table below and FIG. 2. Here, the TOC removal rate was calculated using the formula (750 ppm-TOC)/750 ppm*100.

TABLE 5

| | Galvanic | | | Sludge | | TOC | |
|---|---|---|---|---|---|---|---|
| | Pallet | Stirring | | Dissolution | | Removal | |
| Sample | Content | Time | | Time | TOC | Rate | Remarks |
| 1 | 5 g | 3 | hours | 30 min | 20.8 ppm | 97.2% | Dilution |
| | | | | | | | Analysis |
| 2 | 0.75 g | 30 | min | 30 min | 1.01 ppm | 99.8% | Dilution |
| | | | | | | | Analysis |
| 3 | 0.09 g | 15 | min | 15 min | 20.6 ppm | 97.3% | Original |
| | | | | | | | Solution |
| | | | | | | | Analysis |
| 4 | 0.11 g | 25 | min | 25 min | 21.0 ppm | 97.2% | Original |

Solution
Analysis

FIG. 2 is a photograph related to the measurement of TOC in wastewater using the galvanic pellets. From Table 5 and FIG. 2, it can be seen that the galvanic pellets began to dissolve after 15 minutes of stirring, and by 30 minutes, had transformed into a slurry state. Accordingly, retention time can be 30 minutes or less. Furthermore, the TOC removal rate was 97% or higher, indicating that organic substances causing odors were effectively removed. In the above test, the cylindrical galvanic pellets were used. However, by using granular galvanic pellets and increasing the stirring time, the retention time can be further reduced. In this case, when applying the galvanic pellets to an actual wastewater treatment process, various approaches can be conducted based on the laboratory results, enabling the application of the galvanic pellets in wastewater treatment processes.

### Test Example 5: Plant Growth Effect

The plant growth effect of the galvanic pellet of the present disclosure was verified through lettuce cultivation.

Lettuce seedlings were purchased and cultivated for two weeks, divided into a test group where the galvanic pellets were applied to soil and a comparative group without the application of galvanic pellets.

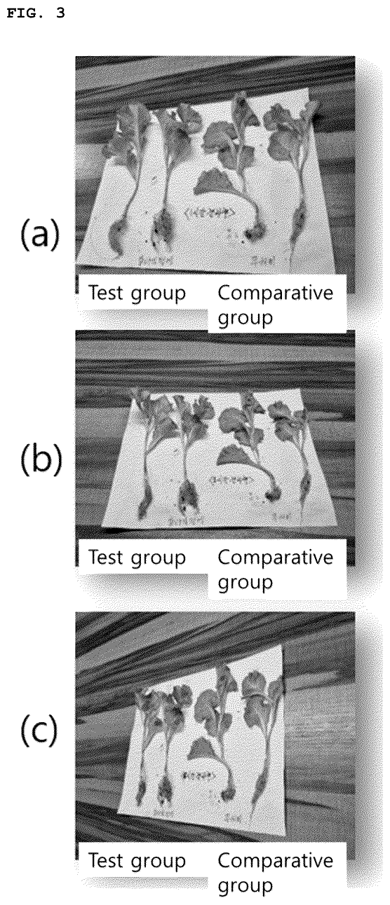

FIG. 3 shows lettuce growth with the application of galvanic pellets, displaying photographs taken after (a) 1 hour of hardening, (b) 3 hours of hardening, and (c) 6 hours of hardening.

From FIG. 3, it can be seen that the amount of roots of the lettuce in the test group is greater than that in the comparative group, indicating that the use of galvanic pellets promotes root growth. Furthermore, when the elasticity of a stem was tested by stretching the stem by hand, the lettuce in the test group showed greater elasticity compared to the lettuce in the comparative group. Furthermore, leaves of the lettuce in the test group were thicker and more robust than those in the comparative group, with neither shrinkage nor curling in the leaves.

As shown in FIG. 3(a) to FIG. 3(c), the condition of the lettuce was observed after being left for a certain period. The lettuce in the test group remained in excellent condition even after 6 hours, whereas the lettuce in the comparative group gradually wilted over time, with its leaves and stems sagging after 6 hours.

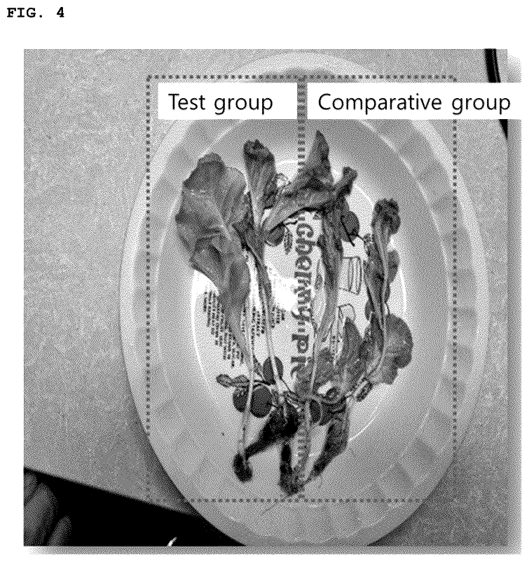

FIG. 4 is a photograph showing the state of lettuce after being washed with water.

From FIG. 4, it can be seen that the lettuce in the test group remained fresh, whereas the lettuce in the comparative group showed wilting in the leaves, stems, and roots.

### INDUSTRIAL APPLICABILITY

A galvanic pellet and a fabrication method thereof according to the present disclosure enable resource e utilization and industrialization by reusing forestry and timber waste, such as sawdust that is conventionally discarded, or livestock manure.

1. A galvanic pellet comprising:
an organic powder; a first metal powder and a second metal powder that have different materials; and a binder,
wherein the first and second metal powders generate microcurrent due to an electric potential difference under presence of an electrolyte.

2. The galvanic pellet of claim 1, wherein the organic powder is obtained by pulverizing one or more types of by-products selected from the group consisting of food waste, livestock waste, woody waste, agricultural by-products, and forestry by-products.

3. The galvanic pellet of claim 1, wherein the first metal powder and the second metal powder each include one selected from Al, Zn, Fe, Ni, Sn, Hg, Ag, Pt, Au, and combinations thereof.

4. The galvanic pellet of claim 1, wherein the binder comprises a biodegradable binder of a single type selected from the group consisting of lignin, starch, collagen, gelatin, hyaluronic acid, chitosan, alginic acid, beta-glucan, cellulose, carrageenan, pluronic, and combinations thereof.

5. The galvanic pellet of claim 1, wherein the galvanic pellet further comprises, as an additive, one selected from the group consisting of a filler, a lubricant, and combinations thereof.

6. The galvanic pellet of claim 5, wherein the filler includes titanium dioxide having photocatalytic activity.

7. The galvanic pellet of claim 1, wherein the galvanic pellet removes odors from pet litter, a food waste disposer, or an adsorbent for a wastewater treatment plant.

8. The galvanic pellet of claim 1, wherein the galvanic pellet is mixed into soil and promotes plant growth.

9. A method of fabricating the galvanic pellet according to claim 1, the method comprising:
mixing an organic powder with a first metal powder and a second metal powder that have different materials, and a binder, and performing a pellet forming process by introducing the mixture into a pellet forming unit,
wherein the first and second metal powders generate microcurrent due to an electric potential difference in presence of an electrolyte.
